(19) United States
(12) Patent Application Publication
(10) Pub. No.: US 2015/0148901 A1
(43) Pub. Date: May 28, 2015

(54) APPARATUS AND METHOD FOR BREAST RECONSTRUCTION AND AUGMENTATION USING AN AUTOLOGOUS PLATELET-RICH FIBRIN MATRIX

(71) Applicant: Anthony Patrick Napolitano, Chappaqua, NY (US)

(72) Inventor: Anthony Patrick Napolitano, Chappaqua, NY (US)

(21) Appl. No.: 14/205,355

(22) Filed: Mar. 11, 2014

Related U.S. Application Data
(60) Provisional application No. 61/780,959, filed on Mar. 13, 2013.

(51) Int. Cl. A61F 2/12; A61L 27/44; A61M 1/00; A61L 27/36

(52) U.S. Cl. CPC A61F 2/12; A61L 27/3616; A61L 27/3641; A61L 27/44; A61M 1/0019; A61L 2430/04; A61M 2202/0413
USPC 623/8; 424/93.72

(57) ABSTRACT

Described herein are systems and methods for soft tissue reconstruction and augmentation using a platelet-rich fibrin matrix. In one embodiment the system of the present invention includes a blood collection apparatus capable of drawing blood from a patient, a matrix preparation container configured to hold a patient's blood while the blood is separated and coagulated to form a platelet-rich fibrin matrix therein, and a matrix delivery device configured to receive the matrix preparation container therein and compress and force the platelet-rich fibrin matrix out of the matrix preparation container and into the patient. In another embodiment the invention described herein is a method in which blood is drawn from a patient and separated and coagulated to form a platelet-rich fibrin matrix. The platelet-rich fibrin matrix is implanted into a patient at the site of a tissue defect or where tissue augmentation is desired.

### CROSS-REFERENCE TO RELATED APPLICATION

This application claims the benefit of the filing date under 35 U.S.C. §119 (e) of U.S. Provisional Application No. 61/780,959, filed Mar. 13, 2013, the contents of which are incorporated by reference in their entirety.

### BACKGROUND OF THE INVENTION

The present invention relates generally to instrumentation, kits and methods for reconstruction or augmentation of soft tissue, including soft tissue such as fat, glands, and blood vessels, and particularly mammary tissue.

Breast cancer often requires women to undergo mastectomies to remove cancerous tissue and prevent metastasis. Removal of significant volumes of breast tissue can leave women physically scarred and emotionally distraught. Many women who have undergone mastectomies may feel insecure about their appearance or feel a loss of their femininity due to the loss of one or both breasts. Therefore many of these women choose to have breast reconstructive surgery.

Elective breast augmentation surgery has also become a prevalent procedure among women seeking to improve their appearance or self-confidence. Both breast reconstruction and augmentation involve surgical implantation of artificial implants which are typically saline or silicone filled polymeric bags. These procedures cause a foreign body reaction within the patient wherein the patients immune cells attempt to wall off the foreign material by encapsulating it in fibrous scare tissue. While these implants may physically replace lost tissue or increase the size of breasts, they do not promote healing or growth of natural tissue. Because they are made from foreign materials they cannot completely mimic the appearance or mechanical properties of natural breasts. Furthermore breast implants may lead to complications such as leakage or hardening that ultimately require additional medical interventions.

### BRIEF SUMMARY OF THE INVENTION

The present invention may generally comprise a system and method of soft tissue reconstruction and augmentation using a platelet-rich fibrin matrix. In most of the below embodiments, the present invention forms a platelet-rich fibrin matrix from components of the patients own blood which can then be implanted into the breast. A platelet-rich fibrin matrix is a blood clot with a decreased number of red blood cells. In certain embodiments the platelet-rich fibrin matrix may be essentially devoid of red blood cells. It comprises platelets within fibrin network and may contain other plasma components and/or white blood cells. Upon implantation this fibrin matrix provides a natural filler material to replace lost tissue or increase breast size. This natural filler has mechanical properties similar to native tissue allowing it to better mimic the look and feel of native breast tissue. Because it is formed from components of the patients own blood that are normally involved in tissue healing, it does not cause a foreign body reaction. After implantation the matrix will begin to degrade and be replaced by new tissue growth. The platelet-rich fibrin matrix provides a temporary scaffold for cells to populate and begin to generate new tissue. As it degrades the platelet-rich fibrin matrix releases growth factors such as platelet derived growth factor (PDGF), fibroblast growth factor (FGF), epithelial growth factor (EGF), vascular endothelial growth factor (VEGF), and transforming growth factor-beta (TGF-β). These growth factors recruit cells from surrounding tissue and cause them to divide ultimately leading to wound healing and the formation of new tissue and blood vessels. In some circumstances formation of significant new tissue may not occur, but the fibrin matrix would still provide a natural filler material to replace lost tissue or increase breast size on a temporary basis.

The present invention may generally, in a first embodiment, include instrumentation for creating a platelet-rich fibrin matrix from a patient's blood and implanting that matrix into the patient's breast. The instrumentation may include any of a blood collection and matrix preparation system, a centrifuge, and a matrix delivery device. The use of these instruments may create implants for increasing breast volume and promoting new tissue growth for breast reconstruction or augmentation surgery. The instrumentation may further include other elements such as sterile packaging (to maintain sterility of the matrix during processing) and centrifuge buckets (for holding the blood collection and matrix preparation system during centrifugation).

In one embodiment, the present invention may include an instrumentation system for breast reconstruction or augmentation; the instrumentation system may include a blood collection and matrix preparation system capable of removing and storing a patient's blood; a centrifuge capable of forming a platelet-rich fibrin matrix from blood; matrix delivery device for implanting the platelet-rich fibrin matrix. The blood collection and matrix preparation system may comprise a blood collection needle, flexible tubing, a blood collection bag, and a large needle-shaped conduit for injection of the platelet-rich fibrin matrix.

The blood collection and matrix preparation system may comprise a blood collection apparatus removably coupled to a matrix preparation container and may be packaged in sterile packaging. The blood collection apparatus may comprise a flexible blood collection tube with a proximal end and a distal end, and a blood collection needle mounted on the distal end. The blood collection needle may be a butterfly needle which may be capped for safety and sterility. The butterfly needle may further be a retractable needle. The matrix preparation container may comprise an ellipsoidal blood collection bag with a large needle-shaped conduit extending radially outward along a major axis of the ellipsoidal bag such that fluid may flow though the conduit and into the bag. The conduit may be elliptical or circular in cross section and may contain a beveled outer tip. The conduit may be rigid or flexible. Additionally, the matrix preparation container may have valves, ports, or the like for removal of red blood cells or platelet-poor plasma. Optionally, the matrix preparation container may also include a physical barrier for sequestering red blood cells to a separate compartment within the matrix preparation container. The barrier may be a valve, a float, a thixotropic gel, beads, a porous membrane, or the like. The proximal end of the blood collection apparatus may further contain an adaptor for removably coupling to and providing fluid communication with the matrix preparation container. Furthermore this adaptor may substantially surround the needle-shaped conduit of the matrix preparation container forming an airtight seal and protecting the sterility of the conduit while the blood collection apparatus is coupled to the matrix preparation container. Certain embodiments may require blood to come into contact with a substance that activates platelets or otherwise supports coagulation while within the matrix preparation container. Such substances may include glass, silica, collagen, or the like. For such embodiments components of the matrix preparation container may be made from, coated with, or otherwise contain such coagulation supporting substances.

The instrumentation system may further include a centrifuge and one or more centrifuge buckets which are configured to safely hold the blood collection and matrix preparation system during centrifugation. The centrifuge buckets may further be shaped to substantially surround the matrix preparation container and maintain its shape during centrifugation.

The instrumentation system may also include a matrix delivery device for implanting the platelet-rich fibrin matrix into a patient. The matrix delivery device may be configured to receive the matrix preparation container therein such that the needle-shaped conduit extends out of one end for insertion into the patient. In a preferred embodiment the matrix delivery device is configured like a gun with a handle and trigger wherein the needle-shaped conduit is substantially aligned with the barrel of the gun and extends outwardly from the distal end of the gun. The trigger may activate actuation means that move a plunger along the axis of the barrel driving the end of the blood collection bag furthest from the conduit toward the conduit thereby forcing the contents of the bag through the conduit and into the patient. The actuation means may be electronic, pneumatic, hydraulic, spring loaded, mechanical, or the like. Alternatively the surgeon may manually push the plunger for example by pushing on the proximal or back end of the gun. In addition to a plunger other means of compressing the blood collection bag may be used.

In an alternate embodiment, the present invention may be a method for creating a platelet-rich fibrin matrix from a patient's blood and implanting that matrix into the patient's breast. The method may include accessing a vein of the patient with a blood collection needle which is attached to a blood collection and matrix preparation system; allowing the blood to flow into and fill the matrix preparation container; centrifuging and coagulating the blood within the blood collection and matrix preparation system to separate red blood cells and form a platelet-rich fibrin matrix; and implanting the platelet-rich fibrin matrix into the patients breast.

The method may further include the step of simultaneously separating red blood cells and coagulating during a single centrifugation. Due to the differing densities of the various blood components it will separate into three layers such that red blood cells are furthest from the axis of rotation of the centrifuge, the platelet-rich fibrin matrix is in an intermediate position, and a platelet poor plasma layer is closest to the axis of rotation. Because this step precludes the use of anticoagulants it requires relatively rapid transfer of the blood collection bag to a centrifuge to prevent the blood from naturally coagulating before red blood cells can be separated from the clot via centrifugation. The blood collection and matrix preparation system may further be placed in a centrifuge bucket configured to receive the matrix preparation container and maintain its shape during centrifugation. The method may also include the step of removing red blood cells from the matrix preparation container via a valve, port or the like. Additionally the blood collection and matrix preparation system may then be placed within a matrix delivery device. In a preferred embodiment the matrix delivery device may take the form of a gun for injection of the platelet-rich fibrin matrix from which the needle-shaped conduit of the matrix preparation container extends outwardly. The gun may further include a plunger, a handle, and a trigger which actuates the plunger which forces the platelet-rich fibrin matrix out through the needle-shaped conduit. The method may further include the step of disconnecting the blood collection apparatus from the matrix preparation container thereby exposing the sterile needle-shaped conduit. In this method the needle-shaped conduit may also be introduced into the patient's breast through an incision in the skin. The platelet-rich fibrin matrix may further be compressed and forced through the conduit and into the patient's breast.

The method my further include the step of preparing the breast to receive the platelet-rich fibrin matrix implant by creating space within the breast for the matrix to reside. The step of preparing the breast may include sharp and/or blunt dissection of the soft tissue within the breast. Additionally the method may include the step of temporarily implanting a tissue expander to stretch and expand the breast. The step of implanting a tissue expander may occur immediately prior to implantation or several weeks beforehand.

An alternative embodiment of the method of the present invention may further include the step of adding an anticoagulant to the blood collection and matrix preparation system to prevent premature clotting of harvested blood. This method may further include the step of introducing a chemical or physical platelet activator or other procoagulant to later reverse the effects of the anticoagulant.

Yet another embodiment of the method of the present invention may further include the steps of adding an anticoagulant to the blood collection and matrix preparation system; separating the red blood cells from the blood to create platelet-rich plasma; and reversing the anticoagulant to form a platelet-rich fibrin matrix from the platelet-rich plasma. Separation of red blood cells from the blood may be performed by any method known in the art including centrifugation, aphaeresis, plasmapheresis, and platelet-rich plasmapheresis. Centrifugation techniques may include the use of a thixotropic separating medium with a density that is less than that of red blood cells but greater than platelets and plasma. Such thixotropic media behave like viscous fluids under the shear forces of centrifugation allowing them to separate along the density gradient and form a physical barrier between the red blood cells and the other blood components. Once removed from the shear forces of the centrifuge the thixotropic medium forms a solid barrier. The anticoagulant may be any anticoagulant known in the art including ethylenediaminetetraacetic acid (EDTA), citrate, oxalate, heparin, and the like. Reversal of the anticoagulant may be accomplished by any method known in the art including the use of proteins or other compounds that activate or catalyze the natural pathways of clotting (“coagulation activators”). These include, for example, thrombin, thromboplastin, calcium (e.g. calcium glucuronate), bismuth compounds (e.g. bismuth subgallate), collagen, desmopressin and analogs, denatured collagen (gelatin), and fibronectin. Vitamin K may contribute to activation of coagulation. This method may also include the step of centrifugation during platelet-rich fibrin matrix formation.

In an alternate embodiment, the present invention may be a method for creating a plurality of platelet-rich fibrin matrixes from a patient's blood and implanting these matrixes into the patient's breast. The method may include puncturing a vein of the patient with a blood collection needle which is attached to a blood collection and matrix preparation system; allowing the blood to flow into and fill the matrix preparation container; centrifuging and coagulation the blood within the blood collection and matrix preparation system to separate red blood cells and form a platelet-rich fibrin matrix; implanting the platelet-rich fibrin matrix into the patients breast; and repeating the previous steps. The repetition of the steps of this method may occur within one treatment such that multiple smaller matrixes are implanted into the patient's breast so that they are adjacent to one another. The handling of smaller volumes of blood may be useful in embodiments where no anticoagulant is used and the blood needs to be centrifuged quickly after collection to avoid premature coagulation. Furthermore the smaller matrix size may allow for smaller incisions and a less invasive procedure. Alternatively the method may be repeated in several treatments over the course of several months. This would allow for more volume to be created as more blood can be safely drawn. Furthermore it allows for the breast tissue to grow more gradually over time.

In an alternate embodiment, the present invention may be a method for creating a platelet-rich fibrin matrix, implanting that matrix into a patient's breast, and separately but concurrently implanting cells into the patient's breast. The implanted cells may add to the initial breast volume as well as increase the number of cells available for new tissue growth. The method may include puncturing a vein of the patient with a blood collection needle which is attached to a blood collection and matrix preparation system; allowing the blood to flow into and fill the matrix preparation container; centrifuging and coagulation the blood within the blood collection and matrix preparation system to form a platelet-rich fibrin matrix; implanting the platelet-rich fibrin matrix into the patient's breast; and injecting cells into the patient's breast. The method may further include the step of obtaining adipocytes, fibroblasts, blood vessel cells, adipose derived stem cells, and the like via liposuction. Additionally the method may include the step of isolating, enriching, or concentrating adipose derived stem cells from liposuction aspirate. Alternatively progenitor or stem cells may be isolated via any other means known in the art.

In an alternate embodiment, the present invention may be a method for creating a platelet-rich fibrin matrix embedded with cells and implanting that matrix into the patient's breast. The embedded cells may add to the initial implant volume as well as increase the number of cells available for new tissue growth. The method may include puncturing a vein of the patient with a blood collection needle which is attached to a blood collection and matrix preparation system; allowing the blood to flow into and fill the matrix preparation container; adding cells to the matrix preparation container; centrifuging and coagulating the blood and cell mixture; and implanting the platelet-rich fibrin matrix with embedded cells into the patient's breast. Alternatively the method may also include the steps of anticoagulating the blood, removing red blood cells, adding cells to the matrix preparation container and reversing the anti-coagulant without further centrifugation. The method may further include the step of obtaining adipocytes, fibroblasts, blood vessel cells, adipose derived stem cells, and the like via liposuction. Additionally the method may include the step of isolating, enriching, or concentrating adipose derived stem cells from liposuction aspirate. Alternatively progenitor or stem cells may be isolated via any other means known in the art.

### BRIEF DESCRIPTION OF THE DRAWINGS

FIGS. 1A-B. illustrates one embodiment of blood collection and matrix preparation system;

FIGS. 2. illustrates a second embodiment of blood collection and matrix preparation system;

FIGS. 3A-B. illustrates a third embodiment of blood collection and matrix preparation system;

FIGS. 4A-B. illustrate a top view of one embodiment of a matrix delivery device which includes a matrix preparation container loaded therein;

FIGS. 5A-C. illustrate a top view of one embodiment of a matrix delivery device which illustrates injection of a platelet-rich fibrin matrix;

FIGS. 6A-B. illustrate a side view of one embodiment of a matrix delivery device which includes a matrix preparation container loaded therein;

FIGS. 7A-B illustrate a side cross-sectional view and top view respectively of one embodiment of a centrifuge to create a platelet-rich fibrin matrix;

FIG. 8 illustrates a top cross-sectional view of one embodiment of a matrix preparation container after formation of a platelet-rich fibrin matrix therein;

FIG. 9 illustrates one embodiment of implantation of a platelet-rich fibrin matrix into a patient.

### DETAILED DESCRIPTION

While the following instrumentation and methods may be used to repair or augment any suitable type of soft tissue—such as adipose, dermis, epithelium, muscle, nerve, or connective tissue in any area of anatomy—reconstruction and augmentation of breast tissue will be the exemplary focus of the disclosure below. As used herein, “proximal” or “proximally” means closer to or towards an operator, e.g., surgeon, while “distal” or “distally” means further from or away from the operator.

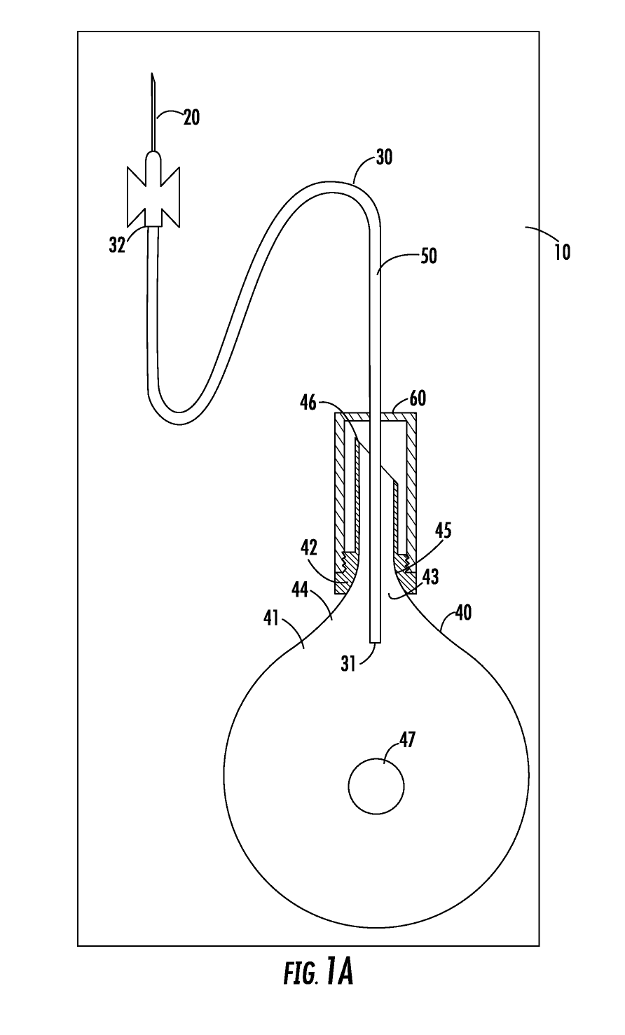

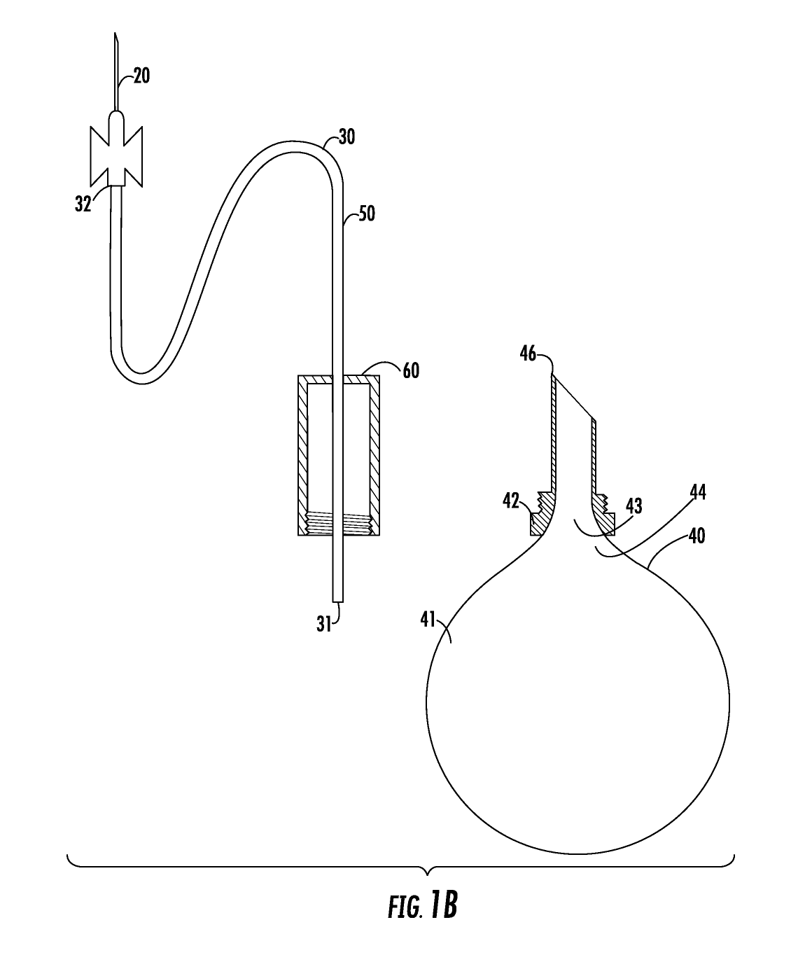

In a first embodiment, the instrumentation system may include a blood collection and matrix preparation system such as those illustrated in FIGS. 1-3. The blood collection and matrix preparation system may comprise a matrix preparation container 40 which is removably attached to a blood collection apparatus 50. Optionally, the blood collection and matrix preparation system may be packaged in a primary sterile package 10. The matrix preparation container may include a blood collection bag 41 and a large needle-shaped conduit 42. In a preferred embodiment the blood collection bag may be generally ellipsoidal in shape and include an opening 43 to which the large needle-shaped conduit is affixed and extends radially outward along a major axis of the ellipsoidal bag such that material may enter or leave the bag through the conduit. The blood collection bag may further include a taper 44 which gradually narrows toward the opening 43 and serves to compress the platelet-rich fibrin matrix as it is forced out of the bag and though the needle-shaped conduit. The conduit may be elliptical or circular in cross section and has a proximal end affixed to the blood collection bag and distal end for insertion into the patient. The conduit may be have a uniform cross-section or may include a taper 45 on the proximal end. The conduit taper 45 may also be substantially continuous with the taper 44 of the blood collection bag serving to further compress the platelet-rich fibrin matrix as it passes through the conduit. The needle-shaped conduit may further include a beveled tip 46 on the distal end. The conduit may be rigid or flexible. Additionally, the matrix preparation container may have valves, ports, or the like for removal of red blood cells or platelet-poor plasma. For example, FIG. 1. illustrates a port 47 on the bottom of the blood collection bag 41. Optionally, the matrix preparation container may also include a physical barrier for sequestering red blood cells to a separate compartment within the matrix preparation container (not shown). The barrier may be a trap door, a valve, a float, a thixotropic gel, beads, a porous membrane, or the like.

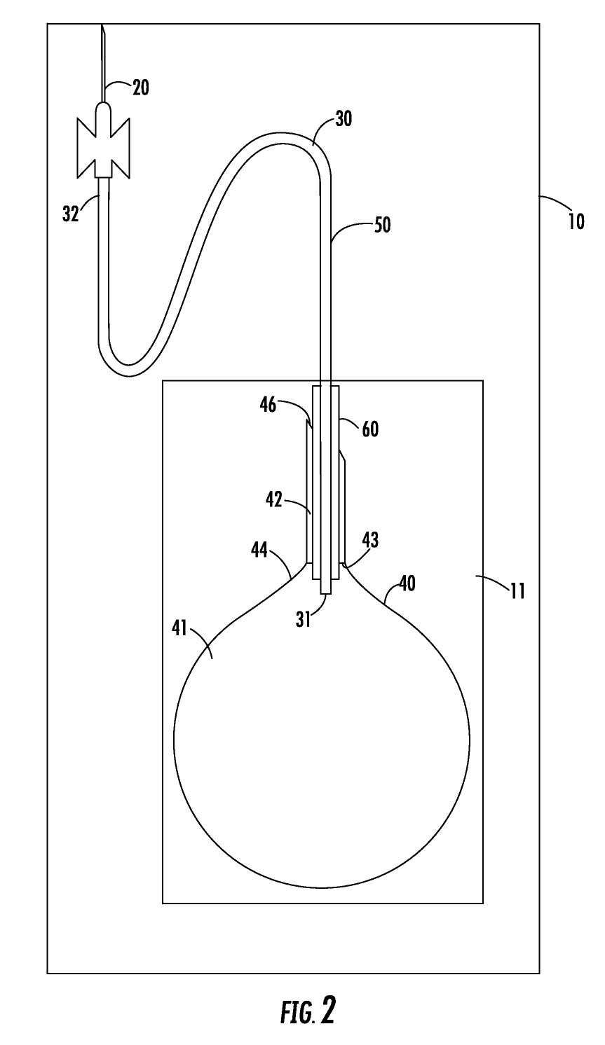

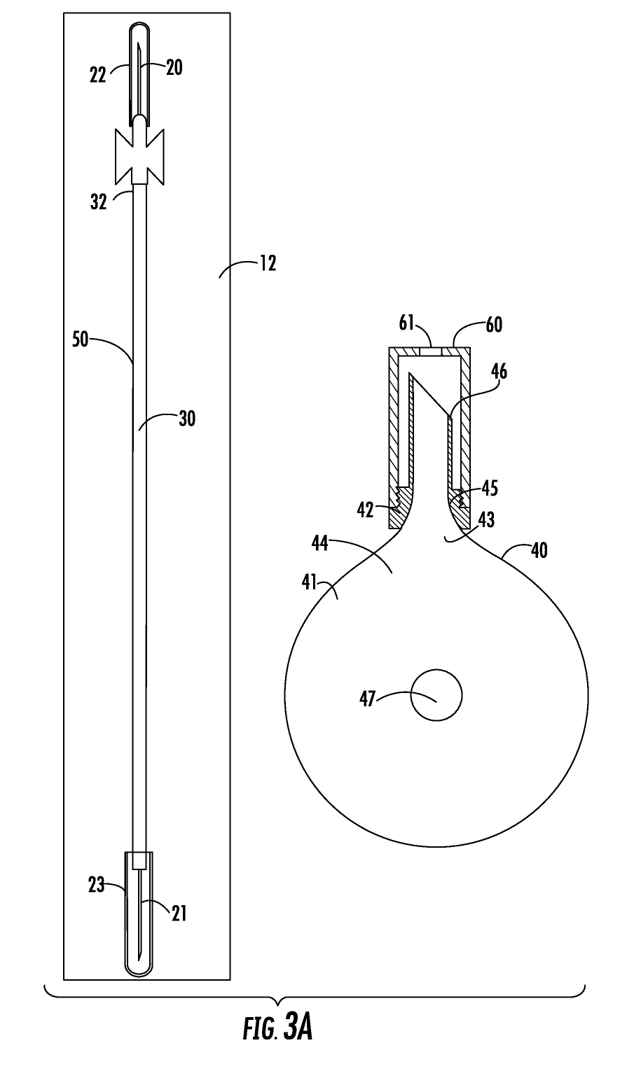

The blood collection apparatus may comprise a flexible blood collection tube 30 with a proximal end 31 and a distal end 32, and a blood collection needle 20 mounted on the distal end. The blood collection needle may be a butterfly needle which may be capped for safety and sterility. The butterfly needle may further be a retractable needle. The proximal end of the blood collection apparatus may further contain an adaptor 60 for removably coupling to and providing fluid communication with the matrix preparation container. FIG. 1. illustrates one embodiment of the adaptor which substantially surrounds the needle-shaped conduit 42 of the matrix preparation container and forms a seal thereby protecting the sterility of the conduit while the blood collection apparatus is coupled to the matrix preparation container. The adaptor may be coupled to the needle-shaped conduit via a threaded, interference fit, frangible or similar connection. FIG. 2. illustrates a second embodiment of adaptor 60 wherein the adaptor is configured to surround the blood collection tube 30 near the proximal end 31 and form an interference fit within the needle-shaped conduit 42. Since this second embodiment of the adaptor 60 does not protect the sterility of the needle-shaped conduit, then a secondary sterile package 11 or the like may be used. When the blood collection apparatus is connected to the matrix preparation container part of the blood collection tube near the proximal end 31 passes through the needle-shaped conduit so that blood may flow through the tube and into the blood collection bag. FIG. 3 illustrates yet another embodiment in which the adaptor is separate from the blood collection apparatus and is removably coupled to the needle-shaped conduit 42. In this embodiment the blood collection tube has a blood transfer needle 21 on the proximal end which is configured to pierce a rubber or elastomeric membrane 61 on the distal end of the adaptor 60 to allow blood to flow through the flexible tubing and into the matrix preparation container. The blood collection needle 20 and blood transfer needle 21 may be covered with a first needle cap 22 and second needle cap 23 respectively.

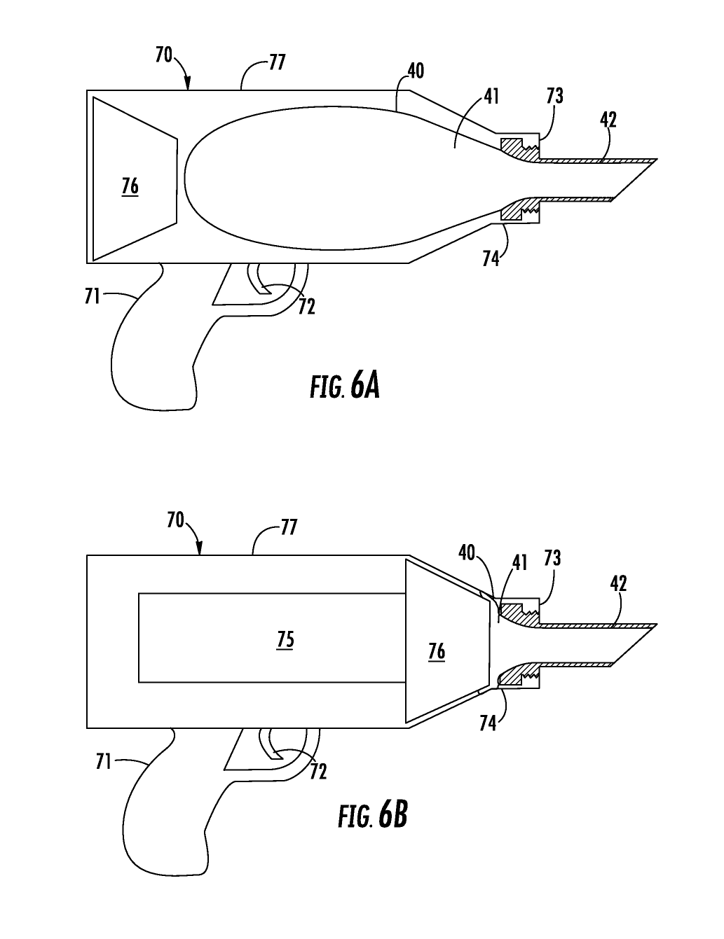

The instrumentation system may further include a centrifuge and one or more centrifuge buckets which are configured to safely hold the blood collection and matrix preparation system during centrifugation. FIGS. 7A-B. illustrate one embodiment of a centrifuge 90 to separate red blood cells and form the platelet-rich fibrin matrix. The centrifuge may have a rotor 91 which is rotated by a motor element 92. The centrifuge further comprises rotor arms 93 that extend radially from the rotor and terminate in centrifuge buckets 94 at the outer end. The centrifuge buckets are configured to receive matrix preparation containers and support them during centrifugation. The centrifuge buckets 94 may further comprise a support element 95 configured to hold the needle-shaped conduit 42 and adaptor 60. The support element may partially or substantially surround the adaptor and/or needle-shaped conduit and may hold the adaptor and/or needle-shaped conduit via a snap fit. In a preferred embodiment the matrix preparation container depicted in FIG. 3 may be placed in the centrifuge after the blood collection tube has been removed but with the adaptor in place to maintain sterility of the needle-shaped conduit 42. The centrifuge embodiment above is described for exemplary purposes as one skilled in the art could design any number of centrifuges that could serve to separate, concentrate, and coagulate blood components; and would be commensurate with the scope of the current invention.

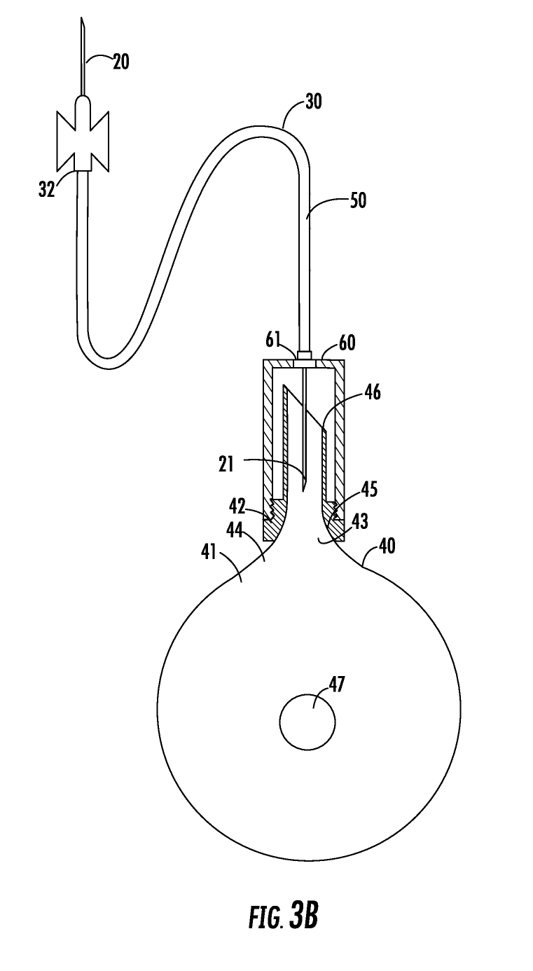

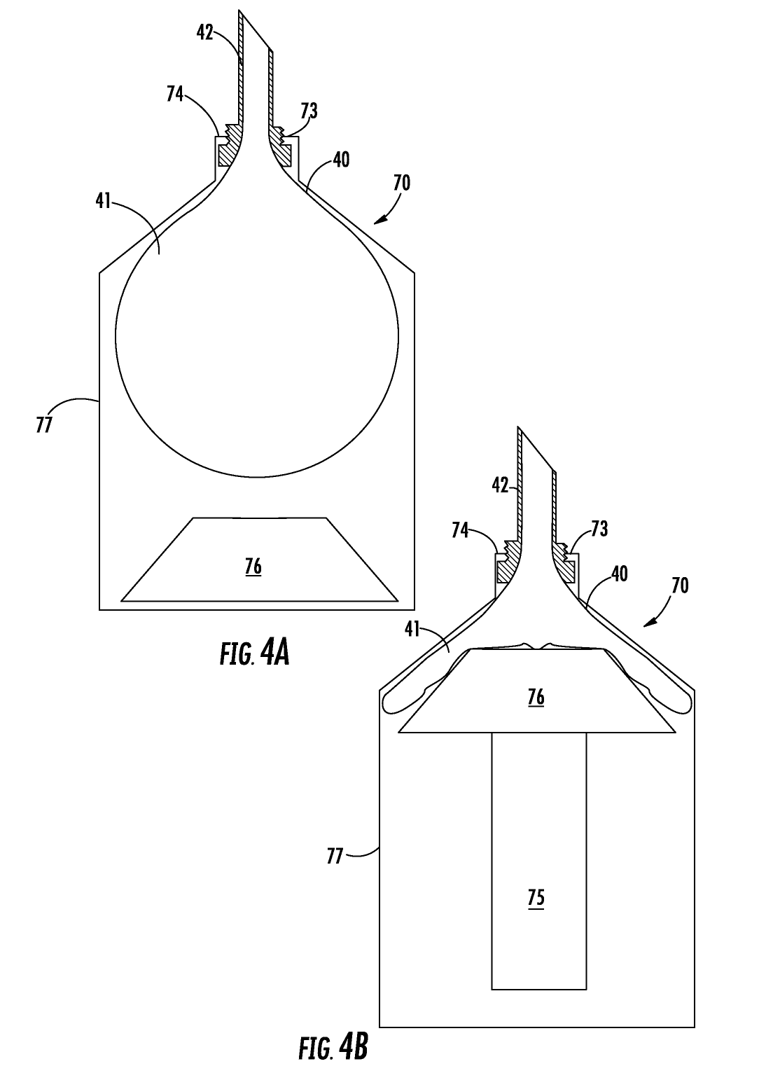

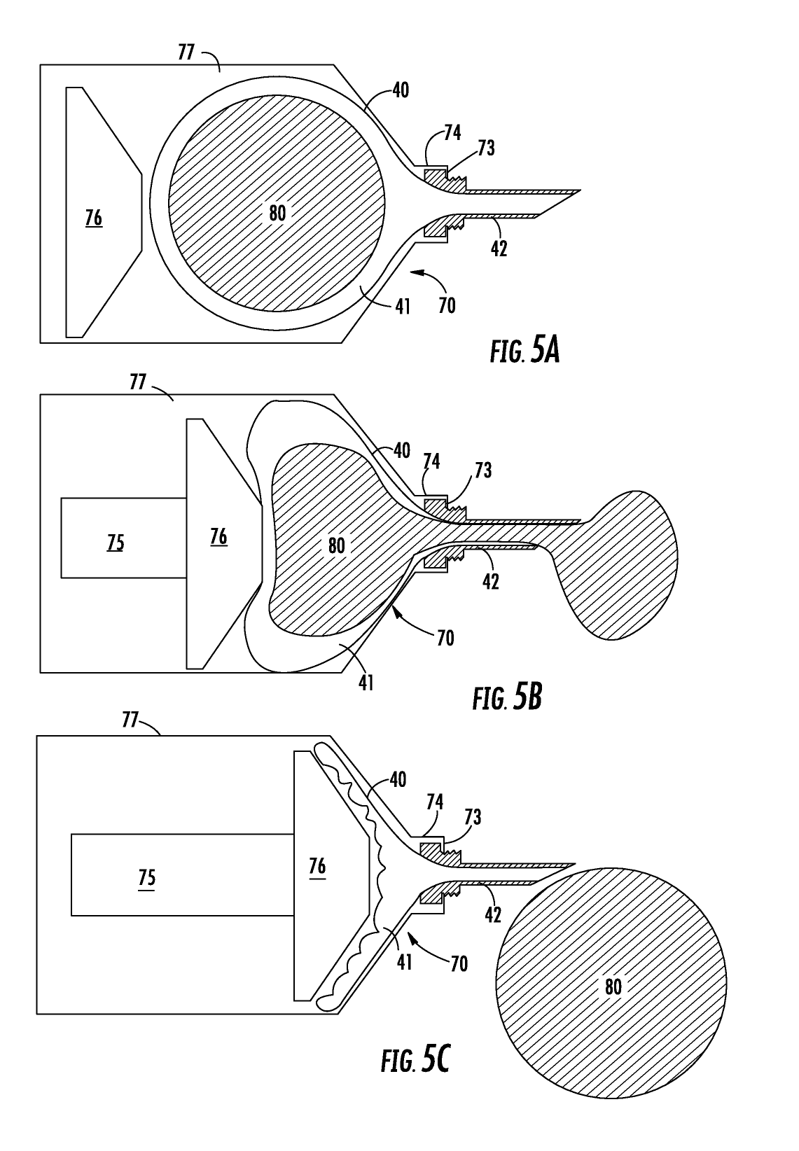

The instrumentation system may also include a matrix delivery device 70 for implanting the platelet-rich fibrin matrix into a patient. The matrix delivery device may be configured to receive the matrix preparation container 40 therein such that the needle-shaped conduit 42 extends out of one end for insertion into the patient. FIGS. 4-6 illustrate a preferred embodiment in which the matrix delivery device is configured like a gun with a barrel 77, handle 71, and trigger 72 wherein the needle-shaped conduit is substantially aligned with the barrel of the gun and extends outwardly from an opening 73 on the distal end 74 of the gun. The trigger activates actuation means 75 that move a plunger 76 along the axis of the barrel 77 from a first position at the proximal end of the gun (as shown in FIGS. 4A, 5A & 6A) to a second position at the distal end of the gun (as shown in FIGS. 4B, 5C & 6B) driving the end of the blood collection bag 41 furthest from the conduit 42 toward the conduit thereby forcing the contents of the bag through the conduit and into the patient. FIG. 5 illustrates a top cross-sectional view of one example in which the platelet-rich fibrin matrix is forced out of the matrix delivery device via the actuation of plunger showing the platelet-rich fibrin matrix 80 prior to activation FIG. 5A, being compressed and forced through the conduit FIG. 5B, and after it has exited the needle-shaped conduit FIG. 5C. The actuation means may be electronic, pneumatic, hydraulic, spring loaded, mechanical, or the like. Alternatively the surgeon may manually push the plunger for example by pushing on the end of a rod that extends from the proximal or back end of the gun (not shown). In addition to a plunger other means of compressing the blood collection bag may be used.

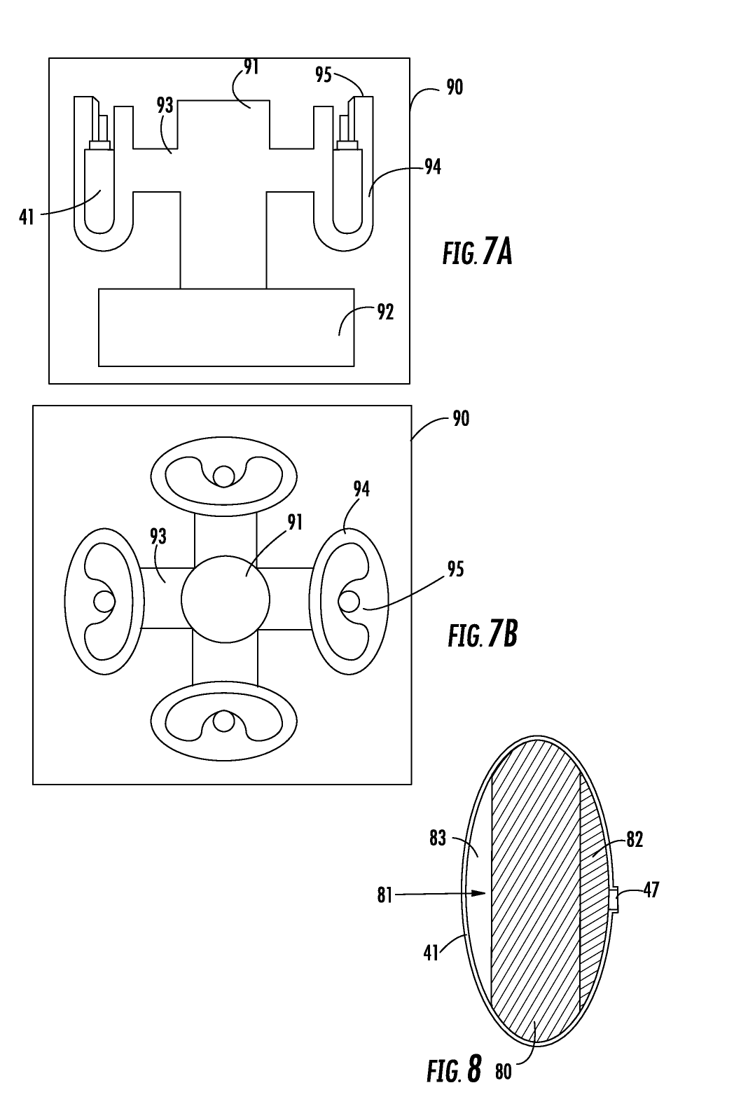

The present invention also includes various methods using the above-discussed instrumentation system for repair, regeneration, or augmentation of soft tissue. As above, the exemplary surgical site will be for implants to increase breast volume and promote new tissue growth for breast reconstruction or augmentation surgery. In a first embodiment, the blood collection and matrix preparation system may be removed from its outer sterile packaging 10 and the blood collection needle 20 used to puncture the vein of a patient. Blood will then flow through the blood collection tube 30 and collect in the matrix preparation container 40. Once blood has filled the matrix preparation container the blood collection needle may be removed from the patient's vein and the blood collection and matrix preparation system may be transferred to a centrifuge. The blood collection and matrix preparation system may be placed into the centrifuge in any position relative to the centrifugal force applied by the centrifuge. In one example illustrated in FIG. 7-8 the major axis of an ellipsoidal blood collection bag 41 may be substantially perpendicular to the direction of centrifugal force 81. Preferably the blood collection and matrix preparation system may be placed in a centrifuge bucket 94 which is configured to support and maintain the shape of the blood collection bag as well as safely hold the needle and tubing during centrifugation.

In another embodiment the blood collection tube 30 of the blood collection apparatus depicted in FIGS. 3A-B may be removed from the sterile packaging 12, the needle cap 23 removed from the blood transfer needle 21, and the needle used to penetrate the membrane 61 of the adaptor 60, the needle cap 22 removed from the blood collection needle 20, and the blood collection needle used to puncture the vein of a patient. Blood will then flow through the blood collection tube 30 and collect in the matrix preparation container 40. Once blood has filled the matrix preparation container the needle may be removed from the patient's vein, the blood transfer needle removed from the adaptor, and the blood collection and matrix preparation system may be transferred to a centrifuge. The blood collection and matrix preparation system may be placed into the centrifuge in any position relative to the centrifugal force applied by the centrifuge. In one example illustrated in FIG. 7-8 the major axis of the ellipsoidal blood collection bag 41 may be substantially perpendicular to the direction of centrifugal force 81. Preferably the blood collection and matrix preparation system may be placed in a centrifuge bucket 94 which is configured to support and maintain the shape of the blood collection bag 41 during centrifugation.

The system may then be centrifuged to simultaneously separate red blood cells and coagulate the blood plasma. To allow for natural coagulation the blood should come into contact with a substance that activates platelets or otherwise supports coagulation while within the matrix preparation container. Such substances may include glass, silica, collagen, or the like. Components of the matrix preparation container 40 may be made from, coated with, or otherwise contain such coagulation supporting substances. The centrifuge may be spun at a slow speed, preferably between 2,000 and 4,000 RPM or between 200 to 1,000 g. Due to the differing densities of the various blood components it will separate into three layers as illustrated in FIG. 8: a red blood cell layer 82 which is furthest from the axis of rotation of the centrifuge, the platelet-rich fibrin matrix 80 which forms in an intermediate position, and a platelet poor plasma layer 83 which is closest to the axis of rotation. Because this particular embodiment of the present invention precludes the use of anticoagulants it requires relatively rapid transfer of the blood collection bag to a centrifuge to prevent the blood from naturally coagulating before red blood cells can be separated from the clot via centrifugation. Optionally, red blood cells and/or platelet poor plasma may be removed from the matrix preparation container via a port 47 or the like.

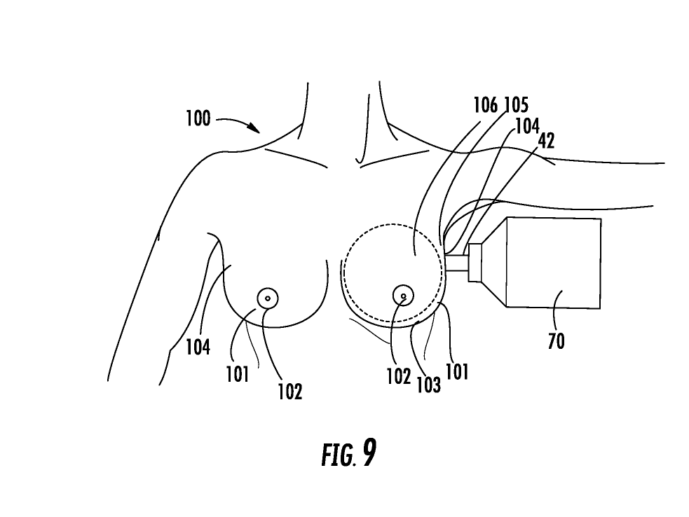

The blood collection and matrix preparation system may then be placed within a matrix delivery device 70. In one example illustrated in FIGS. 4-6 the matrix delivery device may take the form of a gun for injection of the platelet-rich fibrin matrix from which the needle-shaped conduit 42 of the matrix preparation container 40 extends outwardly. The adaptor 60 may be separated from the matrix preparation container 40 thereby exposing the needle-shaped conduit 42. The patient's breast may be prepared for the implantation of the platelet-rich fibrin matrix via any technique known in the art. Typically, a pocket 106, either directly below the breast or below the pectoralis muscle is made by blunt or sharp dissection through a skin incision 105. The pocket can be formed behind the breast tissue or underneath the muscle. Optionally a tissue expander may be used to stretch the skin and soft tissue around the implant location. The optional tissue expander may be implanted into the pocket immediately prior to implantation of the platelet-rich fibrin matrix or within the weeks leading up to the procedure. FIG. 9 illustrates one example in which a matrix delivery device 70 is used to implant a platelet-rich fibrin matrix into a breast 101 of a patient 100. The incision site and surgical approach may be any of those known in the art including for example through incisions around the areola 102, in the infra-mammary fold under the curve of the breast 103, or in the axilla 104. The needle-shaped conduit may then be introduced into the patient's breast through an incision 105 in the skin. Once the needle-shaped conduit is properly placed the matrix delivery device may be activated, for example by pulling a trigger, so the platelet-rich fibrin matrix is compressed and forced through the conduit and into a pocket 106 within the patient's breast. FIG. 5 illustrates one example in which the platelet-rich fibrin matrix is forced out of the matrix delivery device via the actuation of plunger 76 showing the platelet-rich fibrin matrix 80 prior to activation FIG. 5A, being compressed and forced through the conduit FIG. 5B, and after it has exited the needle-shaped conduit FIG. 5C. After implantation of the platelet-rich fibrin matrix the matrix delivery device is removed and the skin incision may be closed.

In a second embodiment, an anticoagulant is included in the blood collection and matrix preparation system in an effective amount to prevent the blood from clotting. Various known anticoagulants may be used including for example ethylenediaminetetraacetic acid (EDTA), citrate, oxalate, heparin and the like. The anticoagulant prevents the blood from naturally coagulating before red blood cells can be separated from the clot via centrifugation thereby affording more time to collect and handle the patient's blood. The blood collection and matrix preparation system may be removed from its outer sterile packaging 10 and the blood collection needle 20 used to puncture the vein of a patient. Blood will then flow through the blood collection tube 30 and collect on the matrix preparation container 40. Once blood has filled the matrix preparation container the blood collection needle may be removed from the patient's vein and the blood collection and matrix preparation system may be transferred to the centrifuge 90. In a preferred embodiment of the blood collection and matrix preparation system 50 depicted in FIG. 3A-B the blood collection tube 30 may be separated from the matrix preparation container 40 by removing the blood transfer needle 21 from the membrane 61 of adaptor 60 before transferring the matrix preparation container 40 into the centrifuge 90. A platelet activator or other procoagulant such as thrombin, collagen, calcium ions, or the like, is introduced, for example through port 47 or membrane 61, to reverse the effects of the anticoagulant. For example, if a calcium chelator such as sodium citrate or EDTA is used as an anticoagulant, it may be reversed with a source of calcium ions such a calcium chloride or calcium gluconate. The blood collection and matrix preparation system may be placed into the centrifuge in any position relative to the centrifugal force applied by the centrifuge. In one example illustrated in FIG. 7-8 the major axis of the ellipsoidal blood collection bag 41 may be substantially perpendicular to the direction of centrifugal force 81. Preferably the blood collection and matrix preparation system may be placed in a centrifuge bucket 94 which is configured to support and maintain the shape of the blood collection bag during centrifugation.

The system may then be centrifuged to simultaneously separate red blood cells and coagulate the blood plasma. The centrifuge may be spun at a slow speed, preferably between 2,000 and 4,000 RPM or between 200 to 1,000 g. Due to the differing densities of the various blood components it will separate into three layers as illustrated in FIG. 8: a red blood cell layer 82 which is furthest from the axis of rotation of the centrifuge, the platelet-rich fibrin matrix 80 which forms in an intermediate position, and a platelet poor plasma layer 83 which is closest to the axis of rotation. Optionally, the separation of red blood cells and coagulation of the plasma may occur in two subsequent steps. In a first example, the blood may be centrifuged before the reversal of the anticoagulant, followed by removal of red blood cells, then addition of the procoagulant and centrifugation to form the platelet-rich fibrin matrix. Red blood cells may be removed from the matrix preparation container via a port 47 or the like. In a second example, the blood may be centrifuged before the reversal of the anticoagulant to create platelet-rich plasma, followed by transfer of the platelet-rich plasma to a new matrix preparation container, then addition of the procoagulant and centrifugation to form the platelet-rich fibrin matrix. In a another example, the blood may be centrifuged before the reversal of the anticoagulant, followed by removal of red blood cells, then addition of the procoagulant to form the platelet-rich fibrin matrix without further centrifugation. In yet another example, plasma and platelets may be collected into the matrix preparation container using a technique that does not permanently remove red blood cells from the patient, such as aphaeresis or platelet-rich plasmapheresis, followed by the addition of the procoagulant and centrifugation to form the platelet-rich fibrin matrix.

The blood collection and matrix preparation system may then be placed within a matrix delivery device 70. In one example illustrated in FIGS. 4-6 the matrix delivery device may take the form of a gun for injection of the platelet-rich fibrin matrix from which the needle-shaped conduit 42 of the matrix preparation container 40 extends outwardly. The adaptor 60 may be separated from the matrix preparation container 40 thereby exposing the needle-shaped conduit 42. The patient's breast may be prepared for the implantation of the platelet-rich fibrin matrix via any technique known in the art. Typically, a pocket 106, either directly below the breast or below the pectoralis muscle is made by blunt or sharp dissection through a skin incision 105. The pocket can be formed behind the breast tissue or underneath the muscle. Optionally a tissue expander may be used to stretch the skin and soft tissue around the implant location. The optional tissue expander may be implanted into the pocket immediately prior to implantation of the platelet-rich fibrin matrix or within the weeks leading up to the procedure. FIG. 9 illustrates one example in which a matrix delivery device 70 is used to implant a platelet-rich fibrin matrix into a breast 101 of a patient 100. The incision site and surgical approach may be any of those known in the art including for example through incisions around the areola 102, in the infra-mammary fold under the curve of the breast 103, or in the axilla 104. The needle-shaped conduit may then be introduced into the patient's breast through an incision 105 in the skin. Once the needle-shaped conduit is properly placed the matrix delivery device may be activated, for example by pulling a trigger, so the platelet-rich fibrin matrix is compressed and forced through the conduit and into a pocket 106 within the patient's breast. FIG. 5 illustrates one example in which the platelet-rich fibrin matrix is forced out of the matrix delivery device via the actuation of plunger 76 showing the platelet-rich fibrin matrix 80 prior to activation FIG. 5A, being compressed and forced through the conduit FIG. 5B, and after it has exited the needle-shaped conduit FIG. 5C. After implantation of the platelet-rich fibrin matrix the matrix delivery device is removed and the skin incision may be closed.

In another embodiment, the present invention may be a method for creating a plurality of platelet-rich fibrin matrixes from a patient's blood and implanting these matrixes into a single breast. The plurality of platelet-rich fibrin matrixes may be produced and implanted in a single treatment session. Alternatively, they may be produced and implanted in multiple treatments over the course of several months. A blood collection and matrix preparation system may be removed from its outer sterile packaging 10 and the blood collection needle 20 used to puncture the vein of a patient. Blood will then flow through the blood collection tube 30 and collect in the matrix preparation container 40. Once blood has filled the matrix preparation container the blood collection needle may be removed from the patient's vein and the blood collection and matrix preparation system may be transferred to a centrifuge. The blood collection and matrix preparation system may be placed into the centrifuge in any position relative to the centrifugal force applied by the centrifuge. In one example illustrated in FIG. 7-8 the major axis of an ellipsoidal blood collection bag 41 may be substantially perpendicular to the direction of centrifugal force 81. Preferably the blood collection and matrix preparation system may be placed in a centrifuge bucket 94 which is configured to support and maintain the shape of the blood collection bag as well as safely hold the needle and tubing during centrifugation.

In another embodiment the blood collection apparatus depicted in FIGS. 3A-B may be removed from the sterile packaging 12, the needle cap 23 removed from the blood transfer needle 21, and the needle used to penetrate the membrane 61 of the adaptor 60, the needle cap 22 removed from the blood collection needle 20, and the blood collection needle used to puncture the vein of a patient. Blood will then flow through the blood collection tube 30 and collect in the matrix preparation container 40. Once blood has filled the matrix preparation container the needle may be removed from the patient's vein, the blood transfer needle removed from the adaptor, and the blood collection and matrix preparation system may be transferred to a centrifuge. The blood collection and matrix preparation system may be placed into the centrifuge in any position relative to the centrifugal force applied by the centrifuge. In one example illustrated in FIG. 7-8 the major axis of the ellipsoidal blood collection bag 41 may be substantially perpendicular to the direction of centrifugal force 81. Preferably the blood collection and matrix preparation system may be placed in a centrifuge bucket 94 which is configured to support and maintain the shape of the blood collection bag 41 during centrifugation.

The system may then be centrifuged to simultaneously separate red blood cells and coagulate the blood plasma. The centrifuge may be spun at a slow speed, preferably between 2,000 and 4,000 RPM or between 200 to 1,000 g. Due to the differing densities of the various blood components it will separate into three layers as illustrated in FIG. 8: a red blood cell layer 82 which is furthest from the axis of rotation of the centrifuge, the platelet-rich fibrin matrix 80 which forms in an intermediate position, and a platelet poor plasma layer 83 which is closest to the axis of rotation. Optionally, red blood cells and/or platelet poor plasma may be removed from the matrix preparation container via a port 47 or the like. If several platelet-rich fibrin matrixes are to be prepared for a single treatment session blood may be drawn into subsequent matrix preparation containers while the previous matrix preparation container is in the centrifuge. In a preferred embodiment depicted in FIG. 3A-B the blood collection needle 20 of the blood collection apparatus 50 can be left in a patient's vein while the blood transfer needle 21 is removed from a first matrix preparation container 40 and connected to subsequent matrix preparation containers. Alternatively, blood access devices such as a Hep-lock or a saline lock may be used so that only one venous puncture can be used to fill multiple matrix preparation containers.

The blood collection and matrix preparation system may then be placed within a matrix delivery device 70. In one example illustrated in FIGS. 4-6 the matrix delivery device may take the form of a gun for injection of the platelet-rich fibrin matrix from which the needle-shaped conduit 42 of the matrix preparation container 40 extends outwardly. The adaptor 60 may be separated from the matrix preparation container 40 thereby exposing the needle-shaped conduit 42. The patient's breast may be prepared for the implantation of platelet-rich fibrin matrixes via any technique known in the art. Typically, a pocket 106, either directly below the breast or below the pectoralis muscle is made by blunt or sharp dissection through a skin incision 105. The pocket can be formed behind the breast tissue or underneath the muscle. Optionally a tissue expander may be used to stretch the skin and soft tissue around the implant location. The optional tissue expander may be implanted into the pocket immediately prior to implantation of the platelet-rich fibrin matrix or within the weeks leading up to the procedure. FIG. 9 illustrates one example in which a matrix delivery device 70 is used to implant a platelet-rich fibrin matrix into a breast 101 of a patient 100. The incision site and surgical approach may be any of those known in the art including for example through incisions around the areola 102, in the infra-mammary fold under the curve of the breast 103, or in the axilla 104. The needle-shaped conduit may then be introduced into the patient's breast through an incision 105 in the skin. Once the needle-shaped conduit is properly placed the matrix delivery device may be activated, for example by pulling a trigger, so the platelet-rich fibrin matrix is compressed and forced through the conduit and into a pocket 106 within the patient's breast. FIG. 5 illustrates one example in which the platelet-rich fibrin matrix is forced out of the matrix delivery device via the actuation of plunger 76 showing the platelet-rich fibrin matrix 80 prior to activation FIG. 5A, being compressed and forced through the conduit FIG. 5B, and after it has exited the needle-shaped conduit FIG. 5C. After implantation of a platelet-rich fibrin matrix the matrix delivery device is removed from the breast. When multiple platelet-rich fibrin matrixes are to be implanted into a single breast, subsequent matrixes are then implanted using the same technique described above wherein the needle-shape conduit 42 is positioned such that the new matrix is implanted adjacent to the previous matrix. Once the last matrix has been implanted the skin incision 105 may be closed. The procedure may also be repeated over the course of several months. This method may be repeated as often as an individual may safely donate blood or blood products. For example a healthy adult may safely donate a pint of blood approximately every eight weeks. It is also envisioned that techniques to collect plasma and platelets that do not remove a patients red blood cells such as aphaeresis may be used to increase the frequency of treatments and volume of matrixes that may safely be used. Although the above method of preparing and implanting a platelet-rich fibrin matrix has been provided as an example, any method described herein is compatible with implanting a plurality of platelet-rich fibrin matrixes into a single breast.

In yet another embodiment, the present invention may be a method for creating a platelet-rich fibrin matrix and implanting that matrix into the patient's breast while separately but concurrently implanting regenerative cells. The implanted cells may add to the initial implant volume as well as increase the number of cells available for new tissue growth. Such cells may also secrete growth factors such as FGF, EGF, VEGF, TGF-β or other cell signaling molecules that promote tissue growth and regeneration. The cells may include connective tissue cells such as adipocytes, fibroblasts, blood vessel cells, adipose derived stem cells, or other progenitor cells. The cells may be autologous, allogenic, or zenogenic. Furthermore the cells may be genetically modified. The cells may be obtained via any technique known to those skilled in the art. For example liposuction may be performed on the patient to collect autologous adipocytes, fibroblasts, blood vessel cells, and adipose derived stem cells to be re-implanted. In another example liposuction aspirate may be processed to enrich the proportion of adipose derived stem cells or isolate adipose derived stem cells to create a substantially pure cell population. By way of nonlimiting example, the liposuction aspirate may be processed via the Icellator Cell Isolation System® (Tissue Genesis, Inc., Honolulu, Hi.) or any method known in the art such as that disclosed in U.S. Pat. No. 8,067,234, the entirety of which is incorporated by reference herein as if fully set forth herein. The blood collection and matrix preparation system may be removed from its outer sterile packaging 10 and the blood collection needle 20 used to puncture the vein of a patient. Blood will then flow through the blood collection tube 30 and collect in the matrix preparation container 40. Once blood has filled the matrix preparation container the blood collection needle may be removed from the patient's vein and the blood collection and matrix preparation system may be transferred to a centrifuge. The blood collection and matrix preparation system may be placed into the centrifuge in any position relative to the centrifugal force applied by the centrifuge. In one example illustrated in FIG. 7-8 the major axis of an ellipsoidal blood collection bag 41 may be substantially perpendicular to the direction of centrifugal force 81. Preferably the blood collection and matrix preparation system may be placed in a centrifuge bucket 94 which is configured to support and maintain the shape of the blood collection bag as well as safely hold the needle and tubing during centrifugation.

In another embodiment the blood collection apparatus depicted in FIGS. 3A-B may be removed from the sterile packaging 12, the needle cap 23 removed from the blood transfer needle 21, and the needle used to penetrate the membrane 61 of the adaptor 60, the needle cap 22 removed from the blood collection needle 20, and the blood collection needle used to puncture the vein of a patient. Blood will then flow through the blood collection tube 30 and collect in the matrix preparation container 40. Once blood has filled the matrix preparation container the needle may be removed from the patient's vein, the blood transfer needle removed from the adaptor, and the blood collection and matrix preparation system may be transferred to a centrifuge. The blood collection and matrix preparation system may be placed into the centrifuge in any position relative to the centrifugal force applied by the centrifuge. In one example illustrated in FIG. 7-8 the major axis of the ellipsoidal blood collection bag 41 may be substantially perpendicular to the direction of centrifugal force 81. Preferably the blood collection and matrix preparation system may be placed in a centrifuge bucket 94 which is configured to support and maintain the shape of the blood collection bag 41 during centrifugation.

The system may then be centrifuged to simultaneously separate red blood cells and coagulate the blood plasma. The centrifuge may be spun at a slow speed, preferably between 2,000 and 4,000 RPM or between 200 to 1,000 g. Due to the differing densities of the various blood components it will separate into three layers as illustrated in FIG. 8: a red blood cell layer 82 which is furthest from the axis of rotation of the centrifuge, the platelet-rich fibrin matrix 80 which forms in an intermediate position, and a platelet poor plasma layer 83 which is closest to the axis of rotation. Because this particular embodiment of the present invention precludes the use of anticoagulants it requires relatively rapid transfer of the blood collection bag to a centrifuge to prevent the blood from naturally coagulating before red blood cells can be separated from the clot via centrifugation. Optionally, red blood cells and/or platelet poor plasma may be removed from the matrix preparation container via a port 47 or the like.

The blood collection and matrix preparation system may then be placed within a matrix delivery device 70. In one example illustrated in FIGS. 4-6 the matrix delivery device may take the form of a gun for injection of the platelet-rich fibrin matrix from which the needle-shaped conduit 42 of the matrix preparation container 40 extends outwardly. The adaptor 60 may be separated from the matrix preparation container 40 thereby exposing the needle-shaped conduit 42. The patient's breast may be prepared for the implantation of the platelet-rich fibrin matrix via any technique known in the art. Typically, a pocket 106, either directly below the breast or below the pectoralis muscle is made by blunt or sharp dissection through a skin incision 105. The pocket can be formed behind the breast tissue or underneath the muscle. Optionally a tissue expander may be used to stretch the skin and soft tissue around the implant location. The optional tissue expander may be implanted into the pocket immediately prior to implantation of the platelet-rich fibrin matrix or within the weeks leading up to the procedure. FIG. 9 illustrates one example in which a matrix delivery device 70 is used to implant a platelet-rich fibrin matrix into a breast 101 of a patient 100. The incision site and surgical approach may be any of those known in the art including for example through incisions around the areola 102, in the infra-mammary fold under the curve of the breast 103, or in the axilla 104. The needle-shaped conduit may then be introduced into the patient's breast through an incision 105 in the skin. Once the needle-shaped conduit is properly placed the matrix delivery device may be activated, for example by pulling a trigger, so the platelet-rich fibrin matrix is compressed and forced through the conduit and into a pocket 106 within the patient's breast. FIG. 5 illustrates one example in which the platelet-rich fibrin matrix is forced out of the matrix delivery device via the actuation of plunger 76 showing the platelet-rich fibrin matrix 80 prior to activation FIG. 5A, being compressed and forced through the conduit FIG. 5B, and after it has exited the needle-shaped conduit FIG. 5C. After implantation of the platelet-rich fibrin matrix the matrix delivery device is removed and the skin incision may be closed.

Although the above method of preparing and implanting a platelet-rich fibrin matrix has been provided as an example, any method described herein is compatible with concurrently implanting regenerative cells. The cells may be implanted via syringe injection into the pocket 106 through the skin incision 105 prior to or subsequent to implantation of the platelet-rich fibrin matrix, or directly through the skin after the incision has been closed. Either a single injection or multiple injections into various locations within the pocket 106 of breast 101 may be used. The cells may be injected as a single-cell suspension in sterile buffered saline or any other pharmaceutically suitable carrier. A single-cell suspension is a suspension of cells in a liquid or aqueous medium wherein substantially all the cells have been isolated so that they are not adhered to other cells. The cells also may be adhered to microcarriers or embedded within microcapsules. Alternatively, the cells may be aggregated into small clusters of cells ranging in diameter from 50 to 500 micrometers, preferably between 100 to 200 micrometers.

In yet another embodiment, the present invention may be a method for creating a platelet-rich fibrin matrix embedded with regenerative cells and implanting that matrix into the patient's breast. The embedded cells may be homogeneously dispersed throughout the platelet-rich fibrin matrix or localized on or near one surface of the platelet-rich fibrin matrix. The embedded cells may add to the initial implant volume as well as increase the number of cells available for new tissue growth. Such cells may also secrete growth factors such as FGF, EGF, VEGF, TGF-β or other cell signaling molecules that promote tissue growth and regeneration. The cells may include connective tissue cells such as adipocytes, fibroblasts, blood vessel cells, adipose derived stem cells, or other progenitor cells. The cells may be autologous, allogenic, or zenogenic. Furthermore the cells may be genetically modified. The cells may be obtained via any technique known to those skilled in the art. For example liposuction may be performed on the patient to collect autologous adipocytes, fibroblasts, blood vessel cells, and adipose derived stem cells to be re-implanted. In another example liposuction aspirate may be processed to enrich the proportion of adipose derived stem cells or isolate adipose derived stem cells to create a substantially pure cell population. By way of nonlimiting example, the liposuction aspirate may be processed via the Icellator Cell Isolation System® (Tissue Genesis, Inc., Honolulu, Hi.) or any method known in the art such as that described in the above-cited '234 patent, incorporated by reference herein.

In a preferred embodiment the embedded cells may be dispersed throughout the platelet-rich fibrin matrix. In this embodiment, the separation of red blood cells and coagulation of the plasma may occur in two subsequent steps. The patient's blood is anticoagulated during collection and separation of red blood cells to prevent the blood from clotting. Various known anticoagulants may be used including for example ethylenediaminetetraacetic acid (EDTA), citrate, oxalate, heparin and the like. The anticoagulant prevents the blood from naturally coagulating before red blood cells can be separated from the blood to create platelet-rich plasma (PRP). Any method known in the art to generate PRP may be used such as those described in U.S. Pat. Nos. 6,979,307, 7,745,106, 6,579,219, 7,553,413, 7,708,152, and 6,398,972, the entireties of which are incorporated by reference herein as if fully set forth herein. For example, a blood collection and matrix preparation system containing an effective amount of anticoagulant to prevent blood clotting may be removed from its outer sterile packaging 10 and the blood collection needle 20 used to puncture the vein of a patient. Blood will then flow through the blood collection tube 30 and collect in the matrix preparation container 40. Once blood has filled the matrix preparation container the blood collection needle may be removed from the patient's vein and the blood collection and matrix preparation system may be transferred to the centrifuge 90. In a preferred embodiment of the blood collection and matrix preparation system 50 depicted in FIG. 3A-B the blood collection tube 30 may be separated from the matrix preparation container 40 by removing the blood transfer needle 21 from the membrane 61 of adaptor 60 before transferring the matrix preparation container 40 into the centrifuge 90. The system may then be centrifuged to separate red blood cells and create PRP. The centrifuge may be spun at a slow speed, preferably between 2,000 and 4,000 RPM or between 200 to 1,000 g. Due to the differing densities of the various blood components it will separate into three layers: a red blood cell layer which is furthest from the axis of rotation of the centrifuge, the buffy coat comprising white blood cells which forms in an intermediate position, and a PRP layer which is closest to the axis of rotation. Red blood cells may be removed from the matrix preparation container, for example, via a port 47 or the like. Alternatively, PRP may be generated using other techniques known in the art and subsequently transferred to the matrix preparation container. In yet another example, plasma and platelets may be collected into the matrix preparation container using a technique that does not permanently remove red blood cells from the patient, such as aphaeresis or platelet-rich plasmapheresis.

Cells may then be added to the matrix preparation container, for example via injection of a single-cell suspension into port 47 or membrane 61. A platelet activator or other procoagulant such as thrombin, collagen, calcium ions, or the like, is introduced, for example through port 47 or membrane 61, to reverse the effects of the anticoagulant. For example, if a calcium chelator such as sodium citrate or EDTA is used as an anticoagulant, it may be reversed with a source of calcium ions such a calcium chloride or calcium gluconate. Mixing means such as rocking, shaking, vibrating, inversion, oscillating, or rotating may be used to keep the cells dispersed throughout the matrix preparation container during coagulation of the platelet-rich fibrin matrix.

In another embodiment the matrix preparation container may be centrifuged after the addition of cells and procoagulant causing the cells to be embedded at an outer surface of the platelet-rich fibrin matrix. The blood collection and matrix preparation system may be placed into the centrifuge in any position relative to the centrifugal force applied by the centrifuge. In one example illustrated in FIG. 7-8 the major axis of the ellipsoidal blood collection bag 41 may be substantially perpendicular to the direction of centrifugal force 81. Preferably the blood collection and matrix preparation system may be placed in a centrifuge bucket 94 which is configured to support and maintain the shape of the blood collection bag 41 during centrifugation.

The centrifuge may be spun at a slow speed, preferably between 2,000 and 4,000 RPM or between 200 to 1,000 g. Most cells are denser than plasma and therefore will become embedded on the surface of the platelet-rich fibrin matrix which is furthest from the axis of rotation of the centrifuge. Cells that are less dense than plasma such as adipocytes will become embedded on the surface of the platelet-rich fibrin matrix which is closest to the axis of rotation.

The blood collection and matrix preparation system may then be placed within a matrix delivery device 70. In one example illustrated in FIGS. 4-6 the matrix delivery device may take the form of a gun for injection of the platelet-rich fibrin matrix from which the needle-shaped conduit 42 of the matrix preparation container 40 extends outwardly. The adaptor 60 may be separated from the matrix preparation container 40 thereby exposing the needle-shaped conduit 42. The patient's breast may be prepared for the implantation of the platelet-rich fibrin matrix via any technique known in the art. Typically, a pocket 106, either directly below the breast or below the pectoralis muscle is made by blunt or sharp dissection through a skin incision 105. The pocket can be formed behind the breast tissue or underneath the muscle. Optionally a tissue expander may be used to stretch the skin and soft tissue around the implant location. The optional tissue expander may be implanted into the pocket immediately prior to implantation of the platelet-rich fibrin matrix or within the weeks leading up to the procedure. FIG. 9 illustrates one example in which a matrix delivery device 70 is used to implant a platelet-rich fibrin matrix into a breast 101 of a patient 100. The incision site and surgical approach may be any of those known in the art including for example through incisions around the areola 102, in the infra-mammary fold under the curve of the breast 103, or in the axilla 104. The needle-shaped conduit may then be introduced into the patient's breast through an incision 105 in the skin. Once the needle-shaped conduit is properly placed the matrix delivery device may be activated, for example by pulling a trigger, so the platelet-rich fibrin matrix is compressed and forced through the conduit and into a pocket 106 within the patient's breast. FIG. 5 illustrates one example in which the platelet-rich fibrin matrix is forced out of the matrix delivery device via the actuation of plunger 76 showing the platelet-rich fibrin matrix 80 prior to activation FIG. 5A, being compressed and forced through the conduit FIG. 5B, and after it has exited the needle-shaped conduit FIG. 5C. After implantation of the platelet-rich fibrin matrix the matrix delivery device is removed and the skin incision may be closed.

1. An instrumentation system for reconstruction and augmentation of soft tissue comprising:
a matrix preparation container configured to hold a patient's blood while said blood is separated and coagulated to form a platelet-rich fibrin matrix therein;
and a matrix delivery device configured to receive said matrix preparation container therein and compress and force said platelet-rich fibrin matrix out of the matrix preparation container and into said patient.

2. The system of claim 1 wherein said matrix preparation container comprises a blood collection bag which includes an opening to which a needle-shaped conduit is affixed and extends outwardly of said blood collection bag such that material may enter or leave the bag through the conduit.

3. The system of claim 2 wherein said blood collection bag is generally ellipsoidal in shape and said needle-shaped conduit extends radially outward along a major axis of said ellipsoidal blood collection bag.

4. The system of claim 3 wherein said blood collection bag further comprises a taper which gradually narrows toward said opening.

5. The system of claim 2 wherein said matrix delivery device is configured to receive said matrix preparation container therein such that said needle-shaped conduit extends out of one end for insertion into the patient.

6. The system of claim 5 wherein said matrix delivery device is configured like a gun with a proximal end, a distal end, and a barrel extending therebetween, a plunger contained within said barrel, an actuation means configured to move said plunger along the axis of said barrel from a first position at the proximal end of the gun to a second position at the distal end of the gun, a handle affixed to said barrel, and a trigger affixed to said barrel adjacent to said handle wherein said trigger activates said actuation means;
and wherein said matrix delivery device is configured to receive said matrix preparation container therein such that said needle-shaped conduit is substantially aligned with the barrel of the gun and extends outwardly from an opening on the distal end of the gun;
and wherein said movement of said plunger along the axis of said barrel from said first position to said second position compresses said blood collection bag.

7. The system of claim 6 further comprising a blood collection apparatus capable of drawing blood from said patient; said blood collection apparatus removably attachable to said matrix preparation container such that blood drawn from said patient may flow from said blood collection apparatus into said matrix preparation container.

8. An instrumentation system for reconstruction and augmentation of soft tissue comprising:
a blood collection apparatus capable of drawing blood from a patient;
a matrix preparation container removably attachable to said blood collection apparatus such that blood drawn from said patient may flow from said blood collection apparatus into said matrix preparation container and be separated and coagulated to form a platelet-rich fibrin matrix therein.

9. The system of claim 8 wherein said matrix preparation container comprises a blood collection bag which includes an opening to which a needle-shaped conduit is affixed and extends outwardly of said blood collection bag such that material may enter or leave the bag through the conduit.

10. The system of claim 9 wherein said blood collection bag is generally ellipsoidal in shape and said needle-shaped conduit extends radially outward along a major axis of said ellipsoidal blood collection bag.

11. The system of claim 10 wherein said blood collection bag further comprises a taper which gradually narrows toward said opening.

12. The system of claim 11 further comprising a matrix delivery device which is configured to receive said matrix preparation container therein such that said needle-shaped conduit extends out of one end for insertion into the patient.

13. The system of claim 12 wherein said matrix delivery device is configured like a gun with a proximal end, a distal end, and a barrel extending therebetween, a plunger contained within said barrel, an actuation means configured to move said plunger along the axis of said barrel from a first position at the proximal end of the gun to a second position at the distal end of the gun, a handle affixed to said barrel, and a trigger affixed to said barrel adjacent to said handle wherein said trigger activates said actuation means;
and wherein said matrix delivery device is configured to receive said matrix preparation container therein such that said needle-shaped conduit is substantially aligned with the barrel of the gun and extends outwardly from an opening on the distal end of the gun;
and wherein said movement of said plunger along the axis of said barrel from said first position to said second position compresses said blood collection bag.

14. A method for reconstruction and augmentation of soft tissue comprising:
separating red blood cells from a patient's blood to create platelet rich plasma;
coagulating the platelet rich plasma within the matrix preparation container of claim 2 to form a platelet-rich fibrin matrix;
and implanting the platelet-rich fibrin matrix into the patient.

15. The method of claim 14 further comprising the steps of:
removably attaching a blood collection apparatus to said matrix preparation container;
accessing a vein of said patient with said blood collection apparatus;
and allowing the patient's blood to flow into and fill the matrix preparation container.

16. The method of claim 15 further comprising the step of placing said matrix preparation container into the matrix delivery device of claim 1 prior to implanting the platelet-rich fibrin matrix into the patient.

17. The method of claim 16 wherein the step of separating red blood cells from said blood comprises centrifugation of said blood.

18. The method of claim 16 further comprising the step of anticoagulating said blood before separation of said red blood cells.

19. The method of claim 16 further comprising the step of implanting cells into said patient.

20. The method of claim 16 wherein said soft tissue comprises human breast tissue.
